(19) United States
(12) Patent Application Publication
(10) Pub. No.: US 2011/0044848 A1
(43) Pub. Date: Feb. 24, 2011

(54) GERMICIDAL APPARATUS AND METHOD FOR STERILIZING OBJECTS

(76) Inventor: Michael Wright, Sandy Spring, MD (US)

Correspondence Address: SUGHRUE MION, PLLC, 2100 PENNSYLVANIA AVENUE, N.W., SUITE 800, WASHINGTON, DC, 20037, US

(21) Appl. No.: 12/516,318

(22) PCT Filed: Dec. 19, 2008
(86) PCT No.: PCT/US08/87594

§ 371 Date: Jan. 15, 2010

Related U.S. Application Data
(60) Provisional application No. 61/016,360, filed on Dec. 21, 2007.

(51) Int. Cl. A61L 2/10; B01J 19/12

(52) U.S. Cl. 422/24; 422/186.3

(57) ABSTRACT

A germicidal apparatus for sterilizing at least one object, including at least one ultraviolet (UV) emitting unit which emits UV light onto at least one object; one or more spraying units which spray at least one chemical onto at least one object, wherein the UV light and at least one chemical sterilize the at least one object. There are disclosed methods for sterilizing at least one object, including a method including emitting ultraviolet (UV) light onto the at least one object; and applying at least one chemical to the at least one object.

This application claims priority to U.S. provisional application Ser. No. 61/016,360 filed Dec. 21, 2007.

### BACKGROUND OF THE INVENTION

1. Field of the Invention

The present invention is directed to a germicidal apparatus and method for sterilizing objects. In particular, the present invention is directed to sterilizing objects in commercial or domestic environments, including, for example, housing facilities and medical environments.

2. Description of the Related Art

Heretofore, numerous methods, apparatuses, and systems have been used to sterilize objects to avoid the spread of infection. However, the related art apparatuses and methods for sterilizing objects consistently fail to provide a means to sterilize objects in a manner that is quick, cost efficient, produces satisfactory results, and is safe and easy to use.

Contaminants, including microorganisms, bacteria, and viruses, are common place in many environments, including commercial or domestic environments, including, for example, housing facilities and medical offices. Contaminants, including microorganisms, bacteria, and viruses, often times settle onto objects that are used, for example, everyday in domestic environments and commercial environments, more particularly in a medical environment, for patient care. Other inventions in the art have been used to control contaminants, including microorganisms, bacteria, and viruses, for example in a medical dental environment; however, such related art methods and apparatus are not time efficient, cost effective, are difficult to use and unreliable.

Regarding commercial environments, examples of medical dentistry related inventions include U.S. Patent Application Pub. No. 2003/0190254, which discloses a method for ultraviolet (UV) disinfecting of compressed air. This method cleans and disinfects compressed air using UV light. The compressed air can then be supplied to the surface of various types of medical and dental instruments. Another apparatus related to sterilization is disclosed by European Patent No. 0463282, which discloses a cabin for the protection and decontamination of a dental arm chair unit. The prior art devices and methods based thereon, and other similar devices, aim to control impurities, such as contaminants, including micro-organisms, bacteria, and viruses, in a medical dental environment.

The above-mentioned devices and methods are discussed to highlight the inventive steps and evolutionary progress of the present invention.

Radiation is known to effect many species population factors in natural, industrial, and domestic ecological systems. The term “radiation” includes all the spectral ranges including the visible spectrum. Radiation at one particular frequency may increase the population of one species of contaminants while simultaneously causing the inactivation or elimination of other species, thereby sterilizing any objects to which the eliminated species was attached.

It is known in the related art that the radiation of UV light can be used to control the presence of bacteria. That is, exposure to UV light (at sufficient flux density and appropriate wave length) kills bacteria and inactivates many organisms or life forms.

UV light is part of the light spectrum, which is classified into three wavelength ranges: UV-C (from wavelength 100 nanometers (nm) to 280 nm); UV-B (from wavelength 280 nm to 315 nm); and UV-A (from wavelength 315 nm to 400 nm). UV-C light is germicidal. UV-C light can influence bacteria, viruses, and other pathogens by destroying their ability to multiply and cause disease. Specifically, UV-C light causes damage to the polynucleotides of microorganisms by forming covalent bonds between adjacent bases in polynucleotides. The formation of such bonds prevents the polynucleotides from being useful for genetic and cellular replication, and thus the organism is unable to reproduce and function. An exemplary list of the microorganisms that can be deactivated by UV-C light is listed in Table 1, herein.

TABLE 1
Exemplary Micro-Organisms Deactivated By
Ultraviolet Germicidal Light
Bacteria

| | |
|---|---|
| Agrobacterium lumefaciens | Pseudomonas aeruginosa |
| Bacillus anthracis | (Environ. Strain) |
| Bacillus anthracis Spores* | Pseudomonas aeruginosa |
| * There are conflicting values reported for inactivation of | (Lab. Strain) |
| the anthrax spore ranging between 9,400-135,000 | Pseudomonas fluorescens |
| microwatt-seconds per square centimeter (for 99% | |
| inactivation). There are some studies underway to | |
| resolve this conflict and we hope to have a reliable value | |
| to report within the next several months. | |
| Bacillus megatherium Sp. (veg) | Rhodospirillum rubrum |
| Bacillus megatherium Sp. (spores) | Salmonella enteritidis |
| Bacillus paratyphosus | Salmonella paratyphi (Enteric Fever) |
| Bacillus subtilis | Salmonella Species |
| Bacillus subtilis Spores | Salmonella typhimurium |
| Clostridium tetani | Salmonella typhi (Typhoid Fever) |
| Clostridium botulinum | Salmonella |
| Corynebacterium diphtheriae | Sarcina lutea |
| Dysentery bacilli | Serratia marcescens |
| Eberthella typhosa | Shigella dysenteriae - Dysentery |
| Escherichia coli | Shigella flexneri - Dysentery |
| Legionella bozemanii | Shigella paradysenteriae |
| Legionella dumoffill | Shigella sonnei |
| Legionella gormanil | Spirillum rubrum |
| Legionella micdadei | Staphylococcus albus |
| Legionella longbeachae | Staphylococcus aureus |
| Legionella pneumophila (Legionnaire's Disease) | Methicillin Resistant Stayphyloccocos |
| | Aureus |
| Leptospiracanicola-Infectious Jaundice | Staphylococcus epidermidis |
| Leptospira interrogans | Streptococcus faecaila |
| Micrococcus candidus | Streptococcus hemolyticus |
| Micrococcus sphaeroides | Streptococcus lactis |
| Mycobacterium tuberculosis | Streptococcus pyrogenes |
| Neisseria catarrhalis | Streptococcus salivarius |
| Phytomonas tumefaciens | Streptococcus viridans |
| Proteus vulgaris | Vibrio comma (Cholera) |
| | Vibrio cholerae |

Molds

| | |
|---|---|
| Aspergillus amstelodami | Oospora lactis |
| Aspergillus flavus | Penicillium chrysogenum |
| Aspergillus glaucus | Penicillium digitatum |
| Aspergillus niger (breed mold) | Penicillium expansum |
| Mucor mucedo | Penicillium roqueforti |
| Mucor racemosus (A & B) | Rhizopus nigricans (cheese mold) |

Protozoa

| | |
|---|---|
| Chlorella vulgaris (algae) | Giardia lamblia (cysts) |
| Blue-green Algae | Nematode Eggs |
| E. hystolytica | Paramecium |

Virus

| | |
|---|---|
| Adeno Virus Type III | Influenza |
| Bacteriophage | Rotavirus |
| Coxsackie | Tobacco Mosaic |
| Infectious Hepatitis | |

Yeasts

| | |
|---|---|
| Baker's Yeast | Saccharomyces cerevisiae |
| Brewer's Yeast | Saccharomyces ellipsoideus |
| Common Yeast Cake | Saccharomyces sp. |

Also, it is well known that some chemicals can kill and inactivate bacteria and other organisms, including mutant and resistant strains thereof, and the chemicals can be used to in part sterilize and decontaminate.

For the purposes of the present invention, the term “sterilizing” can relate to reducing the population of any noxious species (i.e., selective inactivation and/or destruction of disease-causing organisms). The noxious species may be microscopic or macroscopic. Also, the term “sterilizing” can relate to disinfecting or decontaminating objects.

### SUMMARY OF THE INVENTION

In view of the above, an object of the present invention is to provide apparatuses and methods to efficiently and economically sterilize objects in commercial or domestic environments, including, for example, housing facilities and a medical environment, more particularly a dental environment in a thorough manner that is quick, cost efficient, requires little handling of objects to be sterilized, is easy to use and is safe. To achieve the above and other objects, the present invention provides a germicidal apparatus for sterilizing at least one object, said apparatus comprising at least one ultraviolet (UV) emitting unit which emits UV light onto said at least one object; one or more spraying units which spray at least one chemical onto said at least one object, wherein the UV light and said at least one chemical sterilize said at least one object. Also, the present invention provides a germicidal apparatus for sterilizing at least one dental agent, said apparatus comprising at least one ultraviolet (UV) emitting unit which emits UV light onto said at least one dental agent; one or more spraying units which spray at least one chemical onto said at least one dental agent, wherein the UV light and said at least one chemical sterilize said at least one dental agent. An addition to the present invention provides a method for sterilizing at least one object in a germicidal apparatus comprising at least one ultraviolet (UV) emitting unit and one or more spraying units, said method comprising emitting UV light onto said at least one object; and spraying at least one chemical onto said at least one object.

Prior to the present invention however, no invention has been developed that can efficiently and economically sterilize objects in commercial or domestic environments, including, for example, housing facilities and a medical environment, more particularly a dental environment in a thorough manner that is quick, cost efficient, requires little handling of objects to be sterilized, is easy to use and is safe.

### BRIEF DESCRIPTION OF THE DRAWINGS

The attached drawings relate to exemplary and physical embodiments of the present invention.

FIG. 1A displays a germicidal apparatus, including: a (1) power source regulator; (2) radiation regulator; (3) chemical containment chamber; (4) time regulator; (5) table switch; (6) lamp and mounting assembly; (7) radiation chamber door; (8) safety interlock; (9) radiation chamber; (10) chemical jet; (11) reflective interior surface; (12) additional lamp and mounting assembly ports; (13) chemical containment compartments; and (14) chemical port for external containment.

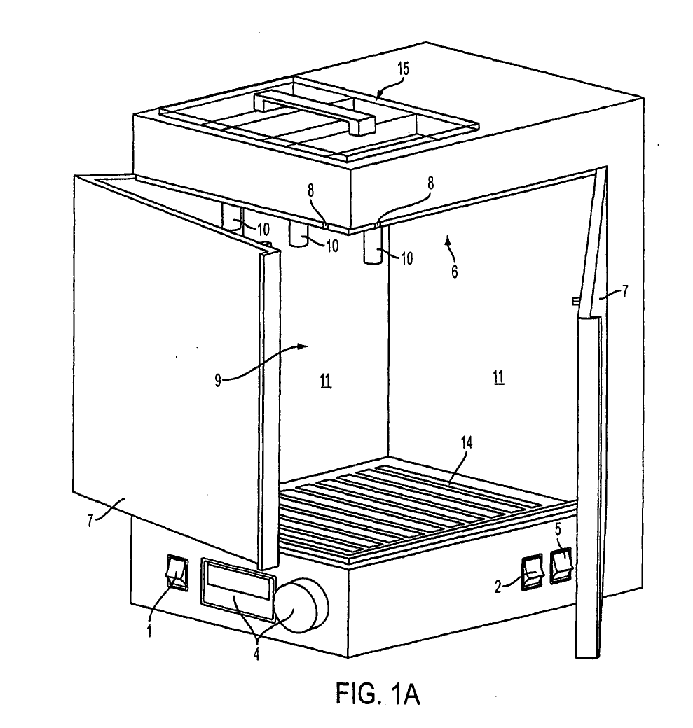

FIG. 1B displays a germicidal apparatus, including: a (1) power source regulator; (2) radiation regulator; (3) plurality of chemical containment chambers; (4) timer regulator; (5) table switch; (6) lamp and mounting assembly; (7) radiation chamber door; (8) safety interlock; (9) radiation chamber; (10) plurality of chemical jets; (11) reflective interior surface; (12) additional lamp and mounting assembly ports; (13) chemical containment compartments; and (14) chemical port for external containment.

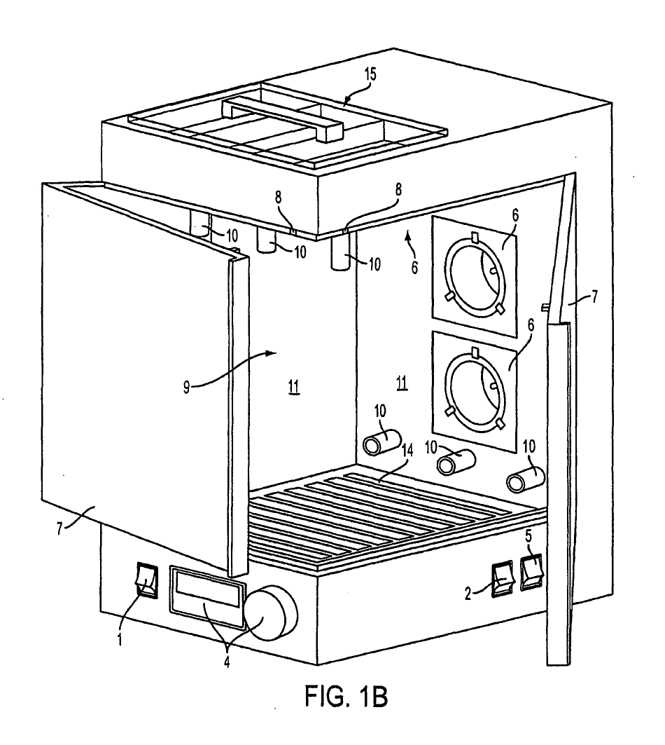

FIG. 1C displays a germicidal apparatus lamp and mounting assembly and chemical jet assembly, including: a (1) lamp access door fastener; (2) lamp mounting; (3) lamp access door; (4) radiation sight line; (5) a nozzle; (6) inner tubular body; (7) outer tubular body; (8) bellows; (9) connector; and (10) positioning device.

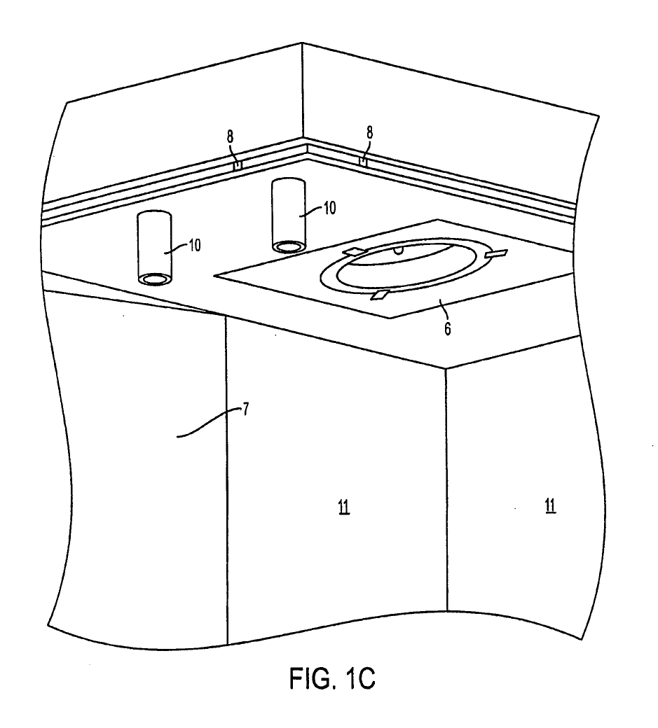

FIG. 1D displays a germicidal apparatus, including: a (1) power source regulator; (2) radiation regulator; (3) plurality of chemical containment chambers; (4) timer regulator; (5) table switch; (6) lamp and mounting assembly; (7) radiation chamber door; (8) safety interlock; (9) radiation chamber; (10) plurality of chemical jets; (11) reflective interior surface; (12) additional lamp and mounting assembly ports; (13) chemical containment compartments; and (14) chemical port for external containment.

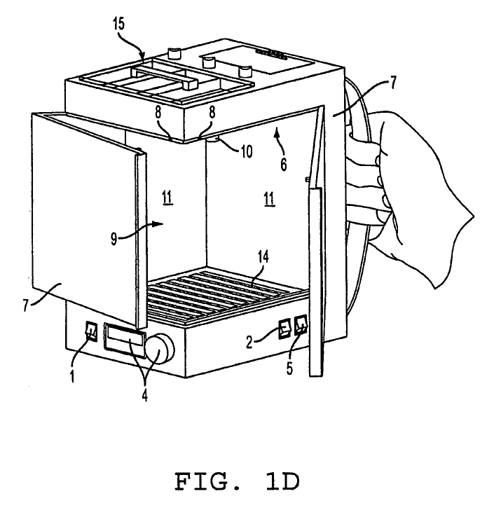

FIG. 1E displays a germicidal apparatus, including: a (1) power source regulator; (2) radiation regulator; (15) plurality of chemical containment chambers; (4) timer regulator; (6) lamp and mounting assembly; (7) radiation chamber door; (9) radiation chamber; (10) plurality of chemical jets; (11) reflective interior surface; (15) chemical containment compartments.

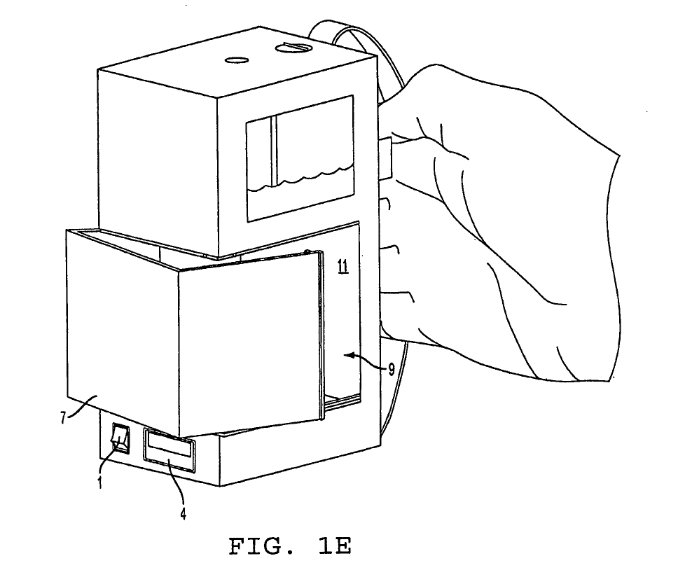

FIG. 2 displays the Colony Forming Units (CFU) obtained following treatment of various contaminated samples including medical mouth mirrors, probes, excavators and protective wear which were swabbed and collected from a clinic and examined in a microbiology laboratory after treatment with a chemical disinfectant, UV c light or a combination. The superior properties of the present invention of synergistic enhanced sterilization are observed.

### DETAILED DESCRIPTION OF THE INVENTION

Certain exemplary embodiments of the present invention will now be described in greater detail.

In an exemplary embodiment of the present invention, a germicidal apparatus for sterilizing objects is provided. The apparatus can be a container in which objects can be placed for sterilization. The container can be self-enclosed and can contain one or a plurality of spraying jet units that can be used to spray one or more chemicals onto an object such that substantially all surfaces of the object can be sprayed with the chemicals. The apparatus may also contain one or more ultraviolet emitting units which emit UV light onto the object or objects contained therein. The emission of the ultraviolet light and the spraying of the chemical can be performed in tandem to sterilize the object that is placed in the germicidal apparatus.

An object can be placed inside of the germicidal apparatus via an opening or doorway in the apparatus.

In an exemplary embodiment of the present invention, the UV light can be emitted simultaneously, or at different times, as the spraying of the chemical, or these operations can be performed sequentially, as further discussed below. The combination of irradiating the UV light and spraying an object with chemical(s) sterilizes the objects to which the UV light and chemical(s) are applied.

As indicated above, the UV light and the one or more chemicals can be applied to the object simultaneously or sequentially. That is, the UV light can be applied to the object first and subsequently the chemical can be applied thereto, or vice versa. Preliminary exemplary results may show that applying the chemical(s) to the object first, and then irradiating the UV light onto the object may produce an optimal result.

The sterilization of the objects placed in the above discussed germicidal apparatus can also be performed for a particular duration of time. That is, the application of the UV light and the chemicals from the spraying units can be applied in a time range of 1 to 7 seconds. This time range, however, can be extended as long as necessary.

The types of chemicals that can be applied using the germicidal apparatus, according to an exemplary embodiment of the present invention, can include various chemicals, including disinfectants, and is not necessarily limited hereto, including ethylene oxide, hydrogen peroxide, sodium chlorite, sodium hypochlorite, alkyl-dimethyl benzyl ammonium chloride, peroxyacetic acid, ethylene glycol monobutyl ether, octanoic acid, benzyl-4-chlorophenol, amylphenol, phenyl phenol, ethyl alcohol, 1-octanaminium, N,N-dimethyl-N-octyl-chloride, 1-decanaminium, N-decyl-N,N-dimethyl-chloride, isopropyl alcohol, thymol, D-gluconic acid, N,N″-bis(4-chlorophenyl)-3,12-diimino-2,4,11,13 tetraazatetradecanediimidamide, dimethyl benzyl ammonium saccharinate, chlorine dioxide, glutaraldehyde, sodium dichloro-s-triazinetrione, poly-ethoxypoly-propoxyethanol-iodine complex, sodium bromide, hypochlorous acid, ethaneperoxoic acid, p-tertiary amylphenol, phosphoric acid, dodecyl sulfonic acid, diethyl toluamide, caprylic acid, didecyl dimethyl ammonium chloride, and sodium dichloro-s-triazinetrione, and combinations, precursors and derivatives thereof and additives thereto.

In an exemplary embodiment of the invention, the duration for irradiating the UV light and applying the one or more chemicals can be determined using a timer that is integrated into the germicidal apparatus.

For example, chemicals for use with the germicidal apparatus of the present invention may exhibit germicidal, fungicidal, virucidal, and tuberculocidal activity within minutes of application at or about room temperature, or other temperatures. Chemicals for use with the germicidal apparatus of the present invention may be a cleaner and or a deodorizer.

A notable aspect of the germicidal apparatus, according to an exemplary embodiment of the present invention, is that a user may no longer personally handle with his/her hand objects that are being sterilized. In the conventional art, objects that are to be sterilized are handled using human hands. The germicidal apparatus according to an exemplary embodiment of the present invention can allow a user a means to insert an object (i.e., tongs, trays and other instruments) into the germicidal apparatus, wherein the germicidal apparatus can perform the operation of sterilizing the object without the need for a user to handle the object with his/her hand, particularly after sterilization has occurred.

In yet another exemplary embodiment of the present invention, the apparatus can include reflective material on the inside thereof for reflecting the irradiated UV light so that substantially all parts of the object that is inserted into the germicidal apparatus will be contacted by the UV light. The reflective materials includes nanoparticles.

In yet another exemplary embodiment of the present invention, the germicidal apparatus can contain single or multiple compartments for holding the various chemicals that will be applied to the object inserted into the germicidal apparatus. This feature permits use of multiple chemicals within a single sterilization.

In yet another exemplary embodiment of the present invention, the germicidal apparatus described above can be portable. The portable germicidal apparatus can be miniature and able to be handheld such that it can be easily transported from one location to another. The portable germicidal apparatus may be powered by batteries or other forms of energy, including, for example, solar energy. Alternatively, the portable germicidal apparatus may actuate a chemical spraying operation using a mechanical means, which may include a push-able mechanism or button.

The portable germicidal apparatus described above can be used to disinfect objects such as, for example, a child's pacifier. That is, a portable handheld battery operated germicidal apparatus which is, for example, 3″h×7″w×2″d in dimension, can be used to disinfect a child's pacifier. A larger germicidal unit can be used to disinfect objects that are larger than a pacifier.

In yet another exemplary embodiment of the present invention, the temperature of the germicidal environment in which an object can be disinfected and/or the temperature of the chemicals, can vary. For example, the temperature of the germicidal environment and/or the chemicals that have been described above can be either at room temperature, below room temperature, or above room temperature. More particularly, the respective temperatures can be either 0-100° C., 20-80° C., 25-65° C., 25-45° C., 25-35° C., or 27° C. The invention may regulate the temperature. Moreover, the amount of chemicals that is applied to an object to be disinfected can vary. The amount of chemicals may include about 1-100 weight percent, 1-90 weight percent, 1-80 weight percent, 1-70 weight percent, 1-60 weight percent, 1-50 weight percent, 1-40 weight percent, 1-30 weight percent, 1-20 weight percent and 1-10 weight percent of each chemical, combinations, precursors and derivatives thereof and additives thereto. The quantity of chemicals applied may include about 1 mg to 1000 g, 10 mg to 100 g, 100 mg to 10 g, 100 mg to 1 g, wherein the quantity applied is an germicidally effective amount.

Yet further, another exemplary embodiment of the present invention can first involve the application of one or more chemicals, and then UV light and chemical(s) from the above-described germicidal apparatus can be applied.

In particular, the present invention is directed to sterilizing objects in commercial or domestic environments, including, for example, residential housing, housing facilities and commercial environments, including medical environments. The invention is useful in all appliances, well known in the art, including refrigeration equipment, freezers, refrigerators, stoves, cookers, ranges, ovens, cooking plates, cooktops, microwave ovens, washing equipment, washing machines, clothes dryers, drying cabinets, dishwashers, air conditioners, water heaters, trash compactors, barbecues, breadmakers, blenders, faucets, food processors, mixers, for example.

### Example

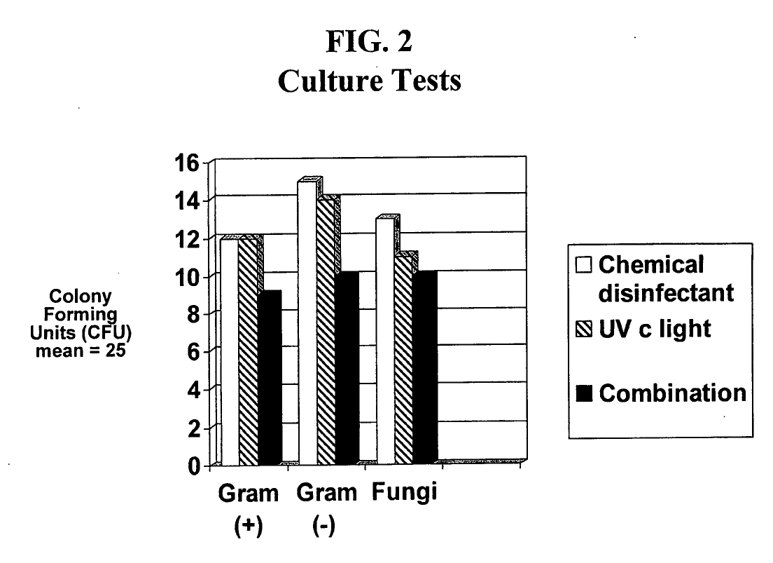

The present invention will now be further explained by way of the following example, which should not be construed as limiting the present invention in any way. Art recognized tests were conducted. Sterilized samples including mouth mirrors, probes, excavators and protective wear were swabbed and collected from a dental clinic and examined in a microbiology laboratory. Samples were cultured using the swab-rinse technique. Sample tubes were incubated at 35 degrees C. for 24 hours and then subcultured on tryptic-digest casein-soy (TS) blood agar aerobically and anaerobically. Colonies were examined by Gram stain and identified by biochemical tests known in the art. Numerous microorganisms were found. The following organisms were further analyzed and subjected to the following sterilization techniques in order to demonstrate the efficacy of the sterilization unit: a) chemical sterilization; b) UV c light; c) combination of chemical sterilization and UV c light. The identified microorganisms were at least: Gram positive (+)ve: Staphylococcus aureus, Staph; Gram negative (−)ve: Pseudomonas aeruginosa: Fungi: Candida albicans. FIG. 2 displays the Colony Forming Units (CFU) obtained following treatment of various contaminated samples including medical mouth mirrors, probes, excavators and protective wear which were swabbed and collected from a clinic and examined in a microbiology laboratory after treatment with a chemical disinfectant, UV c light or a combination. The superior properties of the present invention of synergistic enhanced sterilization are observed.

The foregoing embodiments are merely exemplary and are not to be construed as limiting the present invention. The description of the exemplary embodiments of the present invention is intended to be illustrative, and not to limit the scope of the claims, and many alternatives, modifications, and variations will be apparent to those skilled in the art.

The above described exemplary embodiments can also be part of a larger sterilizing system for sterilizing objects in a multitude of environments. The environments to which exemplary embodiments of the present invention can be applied include any environment in which objects and/or instruments need to be sterilized, decontaminated, and/or purified.

The above-discussed operations and exemplary embodiments thereof can be written as computer programs and can be implemented in general-use digital computers that execute the programs using a computer readable recording medium and other types of transmission media. Examples of the computer readable recording medium include magnetic storage media (e.g., ROM, floppy disks, hard disks, etc.), and optical recording media (e.g., CD-ROMs, or DVDs). Other types of transmission media can include carrier waves (e.g., transmission through the Internet).

Unless otherwise stated, all references and patents cited herein are incorporated by reference.

While the invention has been described in detail with reference to specific embodiments thereof, it will be apparent to one of skill in the art that various changes can be made without departing from the spirit and scope thereof.

What is claimed is:

1. A germicidal apparatus for sterilizing at least one object, said apparatus comprising:
at least one ultraviolet (UV) emitting unit which emits UV light onto said at least one object;
one or more spraying units which spray at least one chemical onto said at least one object,
wherein the UV light and said at least one chemical sterilize said at least one object.

2. The germicidal apparatus according to claim 1, wherein said apparatus comprises a housing in which said at least one object is placed.

3. The germicidal apparatus according to claim 1, wherein said apparatus comprises a plurality of spraying units.

4. The germicidal apparatus according to claim 2, wherein said housing includes an opening via which said at least one object is placed in the housing.

5. The germicidal apparatus according to claim 1, wherein said apparatus further comprises reflective material on the inside of said apparatus, which reflects said emitted UV light.

6. The germicidal apparatus according to claim 5, wherein said reflective material constitutes inner walls of said apparatus.

7. The germicidal apparatus according to claim 3, wherein said plurality of spraying units are disposed within said apparatus such that substantially all surfaces of said at least one object are sprayed with said at least one chemical.

8. The germicidal apparatus according to claim 3, wherein said at least one chemical and said UV light are applied to said at least one object in tandem.

9. The germicidal apparatus according to claim 3, wherein said at least one chemical is first applied to said at least one object, then said UV light is subsequently applied to said at least one object.

10. A germicidal apparatus for sterilizing at least one dental agent, said apparatus comprising:
at least one ultraviolet (UV) emitting unit which emits UV light onto said at least one dental agent;
one or more spraying units which spray at least one chemical onto said at least one dental agent,
wherein the UV light and said at least one chemical sterilize said at least one dental agent.

11. A method for sterilizing at least one object, said method comprising:
emitting ultraviolet (UV) light onto said at least one object; and
applying at least one chemical to said at least one object.

12. The method according to claim 11, wherein said at least one chemical comprises multiple disinfectants.

13. The method according to claim 12, wherein said multiple disinfectants are selected from a group consisting of combinations of isopropyl alcohol, ethylene glycol monobutyl ether and phenyl phenol, phosphoric acid, isopropyl alcohol and dodecyl sulfonic acid.

14. A method for sterilizing at least one object in a germicidal apparatus comprising at least one ultraviolet (UV) emitting unit and one or more spraying units, said method comprising:
emitting UV light onto said at least one object; and
spraying at least one chemical onto said at least one object.

15. The method according to claim 14, wherein said at least one UV emitting unit emits said UV light and said one or more spraying units spray said at least one chemical.

16. The method according to claim 15, wherein said apparatus comprises a plurality of spraying units.

17. The method according to claim 14, wherein said at least one object is inserted in said germicidal apparatus to be sterilized.

18. The method according to claim 16, wherein said apparatus further comprises an opening through which said at least one object is inserted.

19. The method according to claim 14, wherein said apparatus further comprises reflective material on the inside of said germicidal apparatus, which reflect said emitted UV light.

20. The method according to claim 19, wherein said reflective material constitutes inner walls of said germicidal apparatus.

21. The method according to claim 16, wherein said plurality of spraying units are disposed within said apparatus such that substantially all surfaces of said at least one object are sprayed with said at least one chemical.

22. The method according to claim 14, wherein said at least one chemical and said UV light are applied to said at least one object in tandem.

23. The method according to claim 14, wherein said at least one chemical and said emitted UV light are applied one of simultaneously and sequentially.

24. The method according to claim 14, wherein said at least one chemical is first applied to said at least one object, then said UV light is subsequently applied to said at least one object.
